(19) United States
(12) Patent Application Publication
(10) Pub. No.: US 2023/0381251 A1
(43) Pub. Date: Nov. 30, 2023

(54) NOVEL LACTIC ACID BACTERIA ISOLATED FROM AGED MEAT, AND USE THEREOF

(71) Applicant: KOOKMINBIO, CORP., Jeonju-si, Jeollabuk-do (KR)

(72) Inventor: Moon-Hee SUNG, Seoul (KR)

(73) Assignee: KOOKMINBIO, CORP., Jeonju-si, Jeollabuk-do (KR)

(21) Appl. No.: 18/027,614

(22) PCT Filed: Sep. 23, 2021
(86) PCT No.: PCT/KR2021/012927

§ 371 Date: Mar. 21, 2023

(30) Foreign Application Priority Data
Sep. 21, 2020 (KR) 10-2020-0121667
Sep. 17, 2021 (KR) 10-2021-0124768

(51) Int. Cl. A61K 35/747; C12N 1/20; A61P 37/04

(52) U.S. Cl. CPC A61K 35/747; C12N 1/205; A61P 37/04; C12R 2001/25

(57) ABSTRACT

The present invention relates to a Lactiplantibacillus plantarum KM2 strain isolated from aged meat, and a use thereof, wherein the strain has proteolytic activity, immunity-enhancing activity, and probiotic properties, wherein it was found that the Lactiplantibacillus plantarum KM2 strain exhibits proteolytic activity and an immunity-enhancing effect, and does not exhibit hemolytic activity or have genes related to harmful substances or harmful enzymes. Thus, the Lactiplantibacillus plantarum KM2 strain can be used as a material for various probiotics, functional food compositions, and pharmaceutical compositions.

### TECHNICAL FIELD

The present disclosure relates to a Lactiplantibacillus plantarum KM2 strain which has proteolytic activity, immunity-enhancing activity, and probiotic properties and is isolated from aged meat, and a use thereof.

### BACKGROUND ART

Lactic acid bacteria, microorganisms that produce lactic acid using carbohydrates, have long been used industrially, and they enhance flavor and increase nutritional value by decomposing carbohydrates, proteins, and fats through a fermentation process. Since the discovery that the intake of fermented milk that is fermented with Lactobacillus is a factor of longevity, research on functionality of lactic acid bacteria and probiotics has continued. Probiotic lactic acid bacteria are live bacteria that enter the body and exhibit beneficial functionality including an intestinal regulating effect, produce lactic acid in the intestine which becomes acidic to suppress harmful bacteria that cannot withstand acid, create conditions for proliferation of beneficial bacteria which are proliferative in acid to maintain the balance of normal intestinal flora, and support smooth bowel movement, along with reported functions such as immunity enhancement, anticancer actions, and serum cholesterol reduction.

In addition, since various additional efficacies such as lactose intolerance reduction and immune function improvement are expected through the fermentation process, lactic acid bacteria are attracting high interest from consumers and various studies are being conducted therefor. As such, in order to meet product types and consumer preferences that are being diversified, continuous development of superior probiotics is necessary.

Most lactic acid bacteria that are used in Korea are dependent on imports, especially from countries where the dairy industry is developed such as Denmark, the United States, France, and Germany. This may be attributed to the great advancement in improvement technology for lactic acid strains in Western Europe, and the lack of development of lactic acid bacteria that is industrially available in Korea may also be considered as a factor that weakens the competitiveness of lactic acid bacteria.

On the other hand, according to the 2018 Food Yearbook, improvement in living standards and development of medical technology are rapidly accelerating transformation into an aging society, and in Korea, proportion of the elderly population over the age of 65 in 2026 is 20.8% of the total population, predicted that Korea would become a super-aged society that one out of five people is the elderly. As aging progresses in the elderly, physiological functions of tissues and organs constituting the human body irreversibly deteriorate compared to the mature stage, and the risk of chronic degenerative diseases increases with these physical changes, in addition to phenomena such as masticatory disorders, dysphagia, reduction in digestion-absorption rate, and decreased taste. Thereamong, decreased mastication ability may limit the foods to intake while the quantity and quality of diet is downgraded, but fermented milk ingredients may effectively supplement nutrition with ease of ingestion, and in particular, fermentation using lactic acid bacteria with excellent proteolytic properties may provide ease of digestion due to low molecularization of high-molecular substances such as proteins.

### PRIOR ART DOCUMENT

### Patent Document

Korean Patent No. 10-1750948 (published on Jun. 26, 2017)

### DISCLOSURE OF THE INVENTION

### Technical Goals

In order to solve the above problems, an object of the present disclosure is to provide a strain that is applicable as an ingredient for probiotics, functional food compositions, and pharmaceutical compositions since the strain exhibits proteolytic activity and an immunity-enhancing effect without hemolytic activity and is safe due to having no genes related to harmful substances or harmful enzymes.

### Technical Solutions

The present disclosure provides a Lactiplantibacillus plantarum KM2 strain (Accession number: KCTC 14637BP).

In addition, the present disclosure provides a probiotic preparation including the strain, a culture thereof, a fermented product thereof, a concentrate of the culture, a dried product of the culture, or a mixture thereof as an active ingredient.

In addition, the present disclosure provides a composition for enhancing immunity including the strain, a culture thereof, a fermented product thereof, a concentrate of the culture, a dried product of the culture, or a mixture thereof as an active ingredient.

In addition, the present disclosure provides a health functional food composition for enhancing immunity including the strain, a culture thereof, a fermented product thereof, a concentrate of the culture, a dried product of the culture, or a mixture thereof as an active ingredient.

In addition, the present disclosure provides a reagent composition that enhances immunoactivity of immune cells, including the strain, a culture thereof, a fermented product thereof, a concentrate of the culture, a dried product of the culture, or a mixture thereof as an active ingredient.

### Advantageous Effects

According to the present disclosure, it was found that a Lactiplantibacillus plantarum KM2 strain exhibits proteolytic activity and an immunity-enhancing effect without hemolytic activity and has no genes related to harmful substances or harmful enzymes, such that the Lactiplantibacillus plantarum KM2 strain may be applicable as an ingredient for various probiotics, functional food compositions, and pharmaceutical compositions.

### BRIEF DESCRIPTION OF DRAWINGS

FIG. 1 shows results of isolating lactic acid bacteria that produce aged meat-derived proteolytic enzymes and may grow at low temperature.

FIG. 2 shows results of isolating lactic acid bacteria that produce aged meat-derived proteolytic enzymes and may grow at low temperature using a 10% skim milk liquid medium.

FIG. 3 shows a phylogenetic tree designed to identify species names of lactic acid bacteria that produce aged meat-derived proteolytic enzymes and may grow at low temperature.

FIG. 4 is a diagram showing a genetic map of a Lactiplantibacillus plantarum KM2 strain.

FIG. 5 shows results of evaluating hemolytic properties of a Lactiplantibacillus plantarum KM2 strain.

FIG. 6 shows a result of evaluating antibiotic sensitivity of a Lactiplantibacillus plantarum KM2 strain.

FIG. 7 shows results of evaluating an immunity-enhancing effect of a Lactiplantibacillus plantarum KM2 strain.

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

The numerical range includes the numerical value defined in the above range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Hereinafter, the present disclosure will be described in more detail.

As such, the present inventors have completed the present disclosure by discovering that a Lactiplantibacillus plantarum KM2 strain among strains isolated from low-temperature aged meat exhibits proteolytic activity and an immunity-enhancing effect without hemolytic activity and has no genes related to harmful substances or harmful enzymes.

The present disclosure provides a Lactiplantibacillus plantarum KM2 strain (Accession number: KCTC 14637BP).

The Lactiplantibacillus plantarum KM2 strain was deposited in Korean Collection for Type Cultures (KCTC) in Korea Research Institute of Bioscience and Biotechnology on Jul. 14, 2021 and assigned with Accession number KCTC 14637BP.

The strain possesses genes for proteolytic enzymes but not the genes related to harmful substances or harmful enzymes, wherein the proteolytic enzyme is a protease. The protease is an enzyme that hydrolyzes peptide bonds between amino acids constituting a protein with the proteolytic enzyme, some of which include exopeptidases that cleave amino terminus (aminopeptidase) or carboxy terminus (carboxypeptidase) of a protein as well as endopeptidases (e.g., trypsin, chymotrypsin, pepsin, papain, elastase) that cleave the middle of a protein.

The harmful substance and harmful enzymes are derived from acquired antibiotic-resistant genes, virulence genes, and hemolytic genes, and it has been found that the strain does not possess genes encoding such harmful substances and harmful enzymes.

The strain exhibits activity of enhancing immune cell activity, and the immune cells may include macrophages, B cells, T cells, and dendritic cells, and the enhancing of activity of immune cells may be to increase an amount of cytokines secreted by each immune cell for immune responses, specifically, the immune cells are macrophages, and types of cytokines may be TNF-α and INF-β.

In addition, the present disclosure provides a probiotic preparation including the strain, a culture thereof, a fermented product thereof, a concentrate of the culture, a dried product of the culture, or a mixture thereof as an active ingredient.

The probiotics refer to living bacteria, that is, microbes that may inhabit and survive in the gastrointestinal tract when ingested by a person or animal, particularly microbial preparations that are effective in preventing or treating specific pathological conditions. In general, probiotics have an effect of treating and alleviating various symptoms caused by abnormal fermentation of intestinal flora, and when administered to humans and animals, they gather and settle on the wall of the digestive tract in the intestine to prevent harmful microbes from dwelling and produce lactic acid that lowers the pH in the intestine so as to inhibit growth of harmful microbes.

In the probiotic preparation, a culture medium of the strain includes various antibacterial organic acids and non-protein antibacterial substances produced by the strain, and thus exhibits effects comparable to compositions including the strain when included as an active ingredient in probiotic compositions.

The probiotic preparation may be prepared and administered by various formulations and methods according to the methods known in the art. For example, a Lactobacillus plantarum JDFM LP11 strain of the present disclosure, a culture thereof, concentrate of the culture, or dried product thereof may be mixed with carriers commonly used in the pharmaceutical field to be prepared and administered in the form of powder, liquids and solutions, tablets, capsules, syrups, suspensions, or granules. The carrier may be, for example, a binder, lubricant, disintegrant, excipient, solubilizer, dispersant, stabilizer, suspension, color, and fragrance, but is not limited thereto. In addition, the dosage may be appropriately selected according to a degree of absorption of the active ingredient in vivo, an inactivity rate, an excretion rate, the age, sex, species, and condition of a subject, and severity of diseases.

The culture may be an artificial medium obtained by culturing the Lactiplantibacillus plantarum KM 2 strain, wherein the artificial medium may be a commercially prepared synthetic medium to culture lactic acid bacteria and bacteria, such as tryptic soy broth (TBS), tryptic soy broth (TSB), nutrient broth (NB), and Luria-Bertani broth (LB), but is not limited thereto.

The fermented product may be a natural medium fermented using the Lactiplantibacillus plantarum KM 2 strain, wherein the natural medium refers to a natural product that may be fermented with lactic acid bacteria and bacteria, for example, a medium using natural products such as potatoes, tomatoes, and milk, but is not limited thereto.

The concentrate of the culture includes a processed product derived from the culture itself and refers to a material concentrated by performing a process that may be performed as necessary by those skilled in the art, including centrifugation or filtration to collect only bacteria and functional components concentrated by removing a culture medium in the culture.

The dried product of the culture includes a processed product derived from the culture itself and refers to a material dried by performing a process that may be performed as necessary by those skilled in the art, such as centrifugation, filtration, and lyophilization to collect only bacteria and functional components concentrated by removing a culture medium in the culture.

In addition, the present disclosure provides a composition for enhancing immunity including the strain, a culture thereof, a fermented product thereof, a concentrate of the culture, a dried product of the culture, or a mixture thereof as an active ingredient.

The composition for enhancing immunity of the present disclosure may be included in pharmaceutical compositions as needed by those skilled in the art, and the pharmaceutical composition may be prepared in a unit dose form or prepared by infusion in a multi-dose container through formulation using pharmaceutically acceptable carriers according to a method that may be easily carried out by a person skilled in the art to which the present disclosure pertains.

The pharmaceutically acceptable carriers are those commonly used in preparation, and include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and, mineral oil, but are not limited to. The pharmaceutical composition of the present disclosure may further include lubricants, wetting agents, sweetening agents, flavoring agents, emulsifying agents, suspending agents, and preservatives, in addition to the above components.

In the present disclosure, the content of additives included in the pharmaceutical composition is not particularly limited and may be appropriately adjusted within the content range used for conventional preparation.

The pharmaceutical composition may be formulated in the form of one or more external preparations selected from the group consisting of injectable formulations such as aqueous solutions, suspensions, and emulsions, pills, capsules, granules, tablets, creams, gels, patches, sprays, ointments, emplastrum agents, lotions, liniments, pastas, and cataplasmas.

The pharmaceutical composition of the present disclosure may include pharmaceutically acceptable carriers and diluents, which are additional for formulation. The pharmaceutically acceptable carrier and diluent include excipients such as starch, sugar, and mannitol, fillers and extenders such as calcium phosphate, cellulose derivatives such as carboxymethylcellulose and hydroxypropyl cellulose, binders such as gelatin, alginate, and polyvinylpyrrolidone, lubricants such as talc, calcium stearate, hydrogenated castor oil, and polyethylene glycol, disintegrants such as povidone and crospovidone, and surfactants such as polysorbates, cetyl alcohol, and glycerol, but are not limited thereto. The pharmaceutically acceptable carrier and diluent may be biologically and physiologically compatible with subjects. Examples of the diluent may include saline, aqueous buffers, solvents, and/or dispersion media, but are not limited to.

The pharmaceutical composition of the present disclosure may be administered orally or parenterally (e.g., intravenously, subcutaneously, intraperitoneally, or topically) depending on a desired method. For oral administration, the pharmaceutical composition may be formulated as tablets, troches, lozenges, aqueous suspensions, oily suspensions, powder preparation, granules, emulsions, hard capsules, soft capsules, syrups, or elixirs. For parenteral administration, the pharmaceutical composition may be formulated as injections, suppository agents, powder for respiratory inhalation, aerosols for sprays, ointments, powder for application, oil, and creams.

The dosage range of the pharmaceutical composition of the present disclosure may vary depending on the patient's condition, body weight, age, sex, health status, dietary constitution specificity, the nature of preparations, the degree of diseases, administration duration of a composition, administration methods, administration periods or intervals, excretion rate, and drug forms, and may be appropriately selected by those skilled in the art. For example, the dosage may be in the range of about 0.1 to 10,000 mg/kg, but is not limited thereto, while it may be administrated in divided doses from one to several times a day.

The pharmaceutical composition may be administered orally or parenterally (e.g., intravenously, subcutaneously, intraperitoneally, or topically) depending on a desired method. A pharmaceutically effective amount and effective dosage of the pharmaceutical composition of the present disclosure may vary depending on formulation methods, administration methods, administration duration, and/or administration routes of the pharmaceutical composition, and those skilled in the art may easily determine and prescribe the dosage effective for desired treatment. Administration of the pharmaceutical composition of the present disclosure may be conducted once a day or several times in divided doses.

In addition, the present disclosure provides a health functional food composition for enhancing immunity including the strain, a culture thereof, a fermented product thereof, a concentrate of the culture, a dried product of the culture, or a mixture thereof as an active ingredient.

The present disclosure may be generally used as a commonly used food product.

The food composition of the present disclosure may be used as a health functional food. The term “health functional food” as used herein refers to food manufactured and processed with raw materials or ingredients having useful functionality for the human body in accordance with the Health Functional Food Act, and the term “functionality” as used herein refers to the intake to derive effectiveness in health care such as regulation of nutrients or physiological actions for the structure and function of the human body.

The food composition of the present disclosure may include common food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemical compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills.

For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited, and includes all health functional foods in the ordinary sense.

In addition, the present disclosure provides a reagent composition that enhances immunoactivity of immune cells, including the strain, a culture thereof, a fermented product thereof, a concentrate of the culture, a dried product of the culture, or a mixture thereof as an active ingredient.

### MODES FOR CARRYING OUT THE INVENTION

Hereinafter, example embodiments will be described in detail to help the understanding of the present disclosure. However, the following example embodiments are merely illustrative of the content of the present disclosure, and the scope of the present disclosure is not limited to the following examples. The example embodiments of the present disclosure are provided to more completely explain the present disclosure to those of ordinary skill in the art.

### Example 1. Isolation of Lactic Acid Bacteria from Aged Meat

Korean beef that is unaged and Korean beef that was low-temperature aged at 4° C. for 60 days were collected from a restaurant in Gongju City, Chungcheongnam-do. Each beef was mixed with sterile water, crushed, and then diluted stepwise using sterile saline (0.85% NaCl), and 100 μL was taken and smeared on MRS (Lactobacilli MRS agar, Difco) plate medium, followed by culture at 20° C. and 37° C. Colonies formed after the culture were primarily smeared on MRS plate medium to which 0.006% bromocresol purple (BCP, Sigma, St. Louis, MO, USA) which is a pH indicator was added and then cultured at 20° C. and 37° C. for 24 hours to screen 23 species of colonies whose media turned from purple of BCP to yellow as lactic acid bacteria.

As shown in FIG. 1, in order to screen lactic acid bacteria that may grow at low temperatures and produce proteolytic enzymes among the screened lactic acid bacteria, isolated strains were smeared on a plate medium in which BCP and skim milk were mixed and cultured at 4° C. and 20° C. for 24 hours, and 8 species of colonies that may grow at 4° C. and 20° C., become yellow, and form a transparent ring were screened.

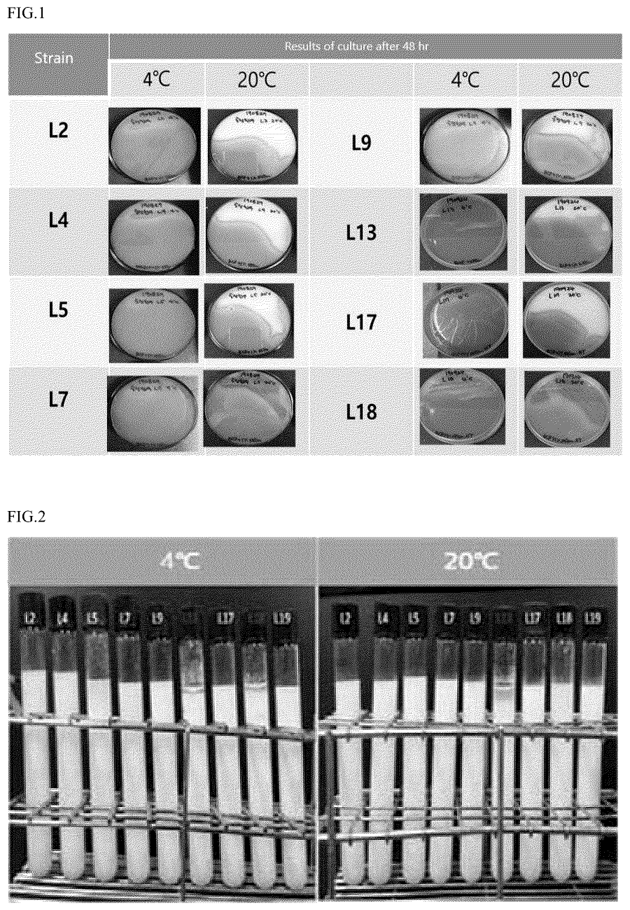

As shown in FIG. 2, in order to screen lactic acid bacteria that produce proteolytic enzymes among the screened 8 species of lactic acid bacteria, culture was performed at 4° C. and 20° C. by tertiary inoculation in 10% skim milk liquid medium, and an L13 strain that coagulates or decomposes skim milk at 4° C. and 20° C. was finally screened.

### Example 2. Identification of the Isolated Strains

For molecular identification and creation of a phylogenetic tree of the strains that were isolated and screened in Example 1, the sequence of 16S rRNA was analyzed by Macrogen, Inc. Sequencing results were compared for homology with sequences registered in GeneBank using nucleotide BLAST from the National Center for Biotechnology Information (NCBI, Bethesda, MD, USA). The sequences of the standard strains were obtained and cross-compared between sequences using the Genetyx program, and then the phylogenetic tree was created using the Kimura 2-parameter method and the neighbor joining algorithm.

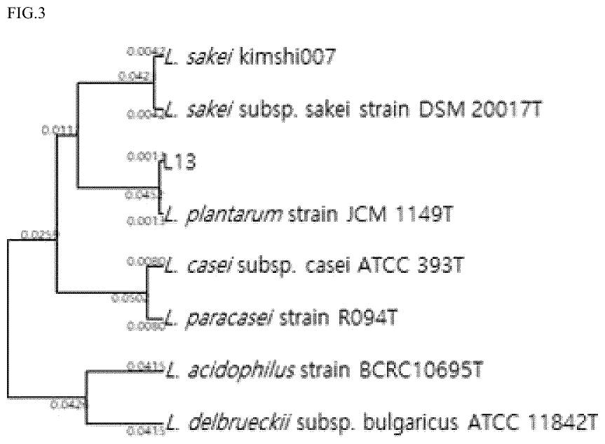

As shown in FIG. 3, the screened L13 strain was identified as Lactobacillus plantarum. The identification process was based on data reclassified for species belonging to the genus Lactobacillus based on genomes in 2020 and was carried out according to the partial revision notice of the standards and criteria for food by the Ministry of Food and Drug Safety on Mar. 30, 2021, and the finally screened L13 strain was named Lactiplantibacillus plantarum KM2 and was deposited in Korean Collection for Type Cultures (KCTC) in Korea Research Institute of Bioscience and Biotechnology on Jul. 14, 2021 with the assigned Accession number KCTC 14637BP.

### Example 3. Verification of Safety of the Isolated Strains in a Genomic Level

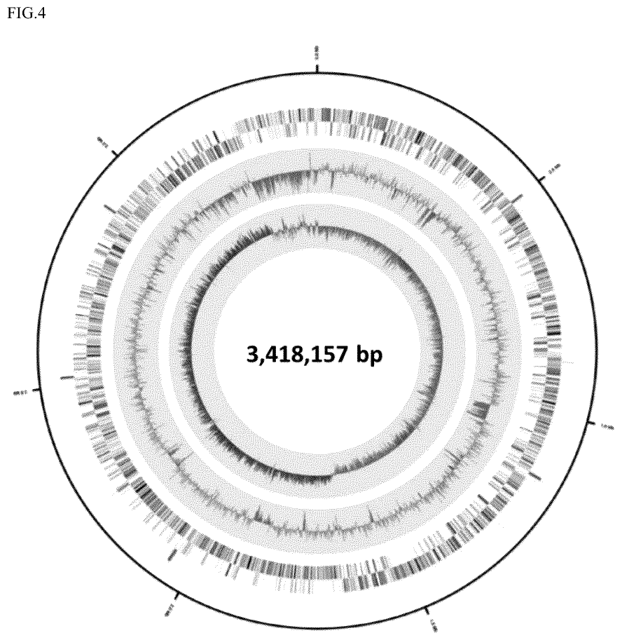

Whole genome sequencing was performed by ChunLab, Inc. in order to determine whether the Lactiplantibacillus plantarum KM2 strain has safety in the genomic level. The genome of the isolated strain was sequenced using PacBio_20K and MiSeq, and the sequencing results were subjected to assembly using MaSuRCA-3.3.9. As shown in FIGS. 4 and Table 1, the result of whole genome sequencing of the Lactiplantibacillus plantarum KM2 strain showed that the strain has 44.24% GC content and 3,418,157 bp of chromosomal DNA and consists of five plasmids. In addition, as shown in Table 2, it was found the strain possesses 13 genes for proteolytic enzymes. In addition, as a result of identifying harmful factors in the genomic level of the Lactiplantibacillus plantarum KM2 strain, it was found that the strain did not possess acquired antibiotic-resistant genes as well as virulence genes and hemolytic factor gene.

TABLE 1

| Features | Value |
|---|---|
| Genome size (bp) | 3,418,157 |
| G + C content (%) | 44.24 |
| Open reading frames | 3,219 |
| CDS assigned by COG | 2,809 |
| rRNA genes | 16 |
| tRNA genes | 69 |

TABLE 2

| Feature name | Product | Length | EC |
|---|---|---|---|
| KM2_00434 | Protease HtpX like protein | 900 | 3.4.24.— |
| KM2_00464 | ATP-dependent zinc | 2238 | 3.4.24.— |
| | metalloprotease FtsH | | |
| KM2_00824 | ATP-dependent Clp protease | 2505 | |
| | ATP-binding subunit ClpC1 | | |
| KM2_00990 | ATP-dependent protease | 744 | |
| KM2_01056 | ATP-dependent Clp protease | 2067 | |
| | ATP-binding subunit ClpA like | | |
| | protein CD4A, chloroplastic | | |
| KM2_01370 | Rhomboid protease | 684 | 3.4.21.105 |
| KM2_01603 | ATP-dependent protease ATPase | 1419 | |
| | subunit HslU | | |
| KM2_01766 | Probable protease eep | 1278 | 3.4.24.— |
| KM2_01884 | ATP-dependent Clp protease | 1266 | |
| | ATP-binding subunit ClpX | | |
| KM2_02065 | Probable inactive | 1266 | 3.4.24.— |
| | metalloprotease YmfF | | |
| KM2_02777 | Putative membrane protease YugP | 699 | |
| KM2_03019 | ATP-dependent Clp protease | 2115 | |
| | ATP-binding subunit ClpL | | |
| KM2_03309 | ATP-dependent protease | 309 | |

### Example 4. Determination of Hemolytic Properties of the Isolated Strains

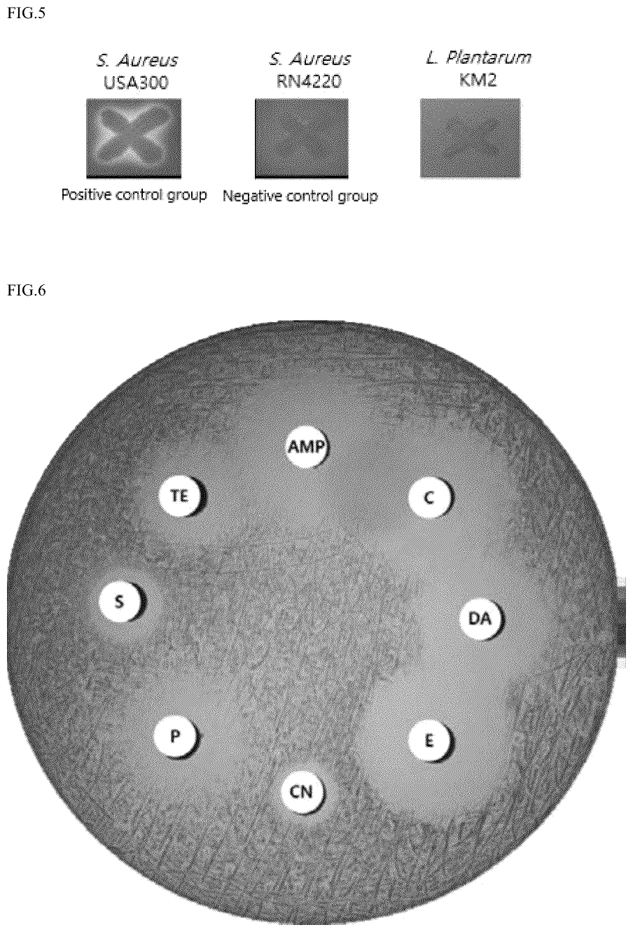

To identify hemolytic properties of the Lactiplantibacillus plantarum KM2 strain, 5% rabbit blood (MB Cell, Korea) or 5% sheep blood (MB Cell, Korea) were added to tryptic soy agar (TSA; BD, Difco) to prepare a blood agar medium. After streaking the Lactiplantibacillus plantarum KM2 strain and culturing at 37° C. for 24 hours, hemolytic properties were identified by formation of transparent rings around the bacteria. A Staphylococcus aureus USA300 strain was used as a positive control group, and a S. aureus RN4220 strain as a negative control group. As shown in FIG. 5, it was found that the Lactiplantibacillus plantarum KM2 strain did not show hemolysis in the blood medium.

### Example 5. Determination of Antibiotic Sensitivity of the Isolated Strains

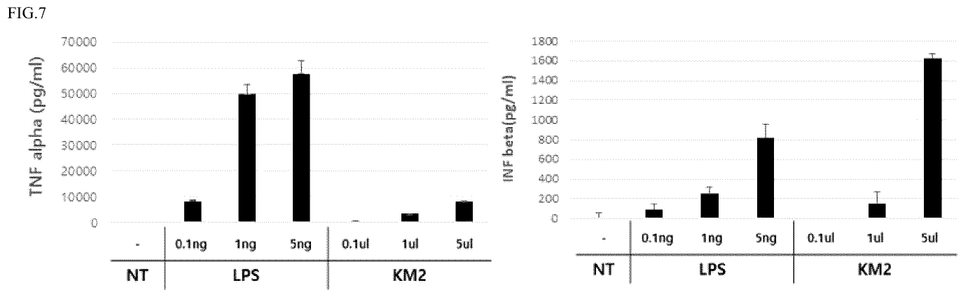

The antibiotic sensitivity of the Lactiplantibacillus plantarum KM2 strain was evaluated using a diffusion method in Muller-Hinton agar medium (BD, Difco) under the guidance of the Clinical and Laboratory Standards Institute (CLSI). Discs containing 8 types of antibiotics were placed on the Muller-Hinton agar medium and cultured at 30° C. for 24 hours. Sizes of inhibition zones for each antibiotic were measured, and sensitivity and resistance were checked by the Clinical Laboratory Standards Institute (CLSI). 8 types of antibiotics were used, including ampicillin (10 ug), chloramphenicol (30 ug), clindamycin (10 ug), erythromycin (15 ug), gentamicin (30 ug), penicillin G (10 ug), streptomycin (300 ug), and tetracycline (30 ug) from Oxoid (Basingstoke, Hants, UK). As shown in FIG. 6, it was found that the Lactiplantibacillus plantarum KM2 strain showed sensitiveness to the 8 types of antibiotics including ampicillin, chloramphenicol, clindamycin, erythromycin, gentamicin, penicillin G, streptomycin, and tetracycline.

### Example 6. Evaluation on Immunity-Enhancing Activity of the Isolated Strains

To identify an immunity-enhancing effect of the Lactiplantibacillus plantarum KM2 strain, evaluation was performed on an effect of the Lactiplantibacillus plantarum KM2 strain on cytokine secretion by immune cells.

Mouse macrophages (RAW 264.7) were used to measure the secretion ability of tumor necrosis factor-α (TNF-α) which is a cytokine that plays an important role in the initial immune response as well as interferon-β (INF-β) that plays an important role in the protective immune response against viral and microbial infections. A culture supernatant of the Lactiplantibacillus plantarum KM2 strain was diluted to a final concentration of 4% using DMEM medium containing 1% fetal bovine serum (FBS) and 1% antibiotics, treated on cells, and cultured in an incubator at 37° C. in the presence of 5% CO₂ for 24 hours. Amounts of TNF-α and INF-β secreted to the supernatant after the culture were measured using an OptEIA™ ELISA kit (BD Biosciences, SanDiego, CA, USA). Absorbance of the measured sample was measured at 450 nm using a SpectraMax 190 Microplate Reader (Molecular Devices, US). O.D values obtained by measuring the absorbance at 450 nm was compared with a standard curve created by using a standard material to measure the amounts of TNF-α and INF-β. As the standard material, recombinant mouse TNF-α and INF-β were used, and calculation was performed by comparing with the obtained standard curve. As a positive control material for TNF-α and INF-β secretion, lipopolysaccharide (LPS) was used.

As shown in FIG. 7, when the culture supernatant of the Lactiplantibacillus plantarum KM2 strain was treated to macrophages, it was observed that the secretion amounts of TNF-α and INF-β, which are cytokines secreted by macrophages, increased depending on a treated concentration of the supernatant.

As described above, a specific part of the content of the present disclosure is described in detail, for those of ordinary skill in the art, it is clear that the specific description is only a preferred embodiment, and the scope of the present disclosure is not limited thereby. In other words, the substantial scope of the present disclosure may be defined by the appended claims and their equivalents.

1. A Lactiplantibacillus plantarum KM2 strain (Accession number: KCTC 14637BP).

2. The Lactiplantibacillus plantarum KM2 strain of claim 1, wherein the strain possesses genes for proteolytic enzymes.

3. The Lactiplantibacillus plantarum KM2 strain of claim 1, wherein the strain enhances activity of immune cells.

4-7. (canceled)

8. A method of enhancing immunity, comprising:
administering a probiotic composition comprising the strain of claim 1, a culture thereof, a fermented product thereof, a concentrate of the culture, a dried product of the culture, or a mixture thereof as an active ingredient to a subject.

9. The method of claim 8, wherein the strain possesses genes for proteolytic enzymes.

10. The method of claim 8, wherein the strain enhances activity of immune cells.

11. A method of enhancing immunity, comprising:
administering a health functional food composition comprising the strain of claim 1, a culture thereof, a fermented product thereof, a concentrate of the culture, a dried product of the culture, or a mixture thereof as an active ingredient to a subject.

12. The method of claim 11, wherein the strain possesses genes for proteolytic enzymes.

13. The method of claim 11, wherein the strain enhances activity of immune cells.

14. A method of enhancing immunoactivity of immune cells, comprising:
administering a reagent composition comprising the strain of claim 1, a culture thereof, a fermented product thereof, a concentrate of the culture, a dried product of the culture, or a mixture thereof as an active ingredient to a subject.

15. The method of claim 14, wherein the strain possesses genes for proteolytic enzymes.

16. The method of claim 14, wherein the strain enhances activity of immune cells.
